(19) United States
(12) Patent Application Publication
(10) Pub. No.: US 2008/0159976 A1
(43) Pub. Date: Jul. 3, 2008

(54) Methods for lowering elevated uric acid levels using intravenous injections of PEG-uricase

(76) Inventors: Jacob Hartman, Holon (IL); Simona Mendelovitz, Ramat Aviv (IL); Claudia D. Rehrig, Plainsboro, NJ (US); William Huang, Florham Park, NJ (US); Zebulun D. Horowitz, Basking Ridge, NJ (US); Royce W. Waltrip, Cranbury, NJ (US); David Erick Wright, Ramona, CA (US); Michelle A. Alton, Yardley, PA (US); Allan N. Maroli, Belle Mead, NJ (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 11/899,688

(22) Filed: Sep. 7, 2007

Related U.S. Application Data
(63) Continuation-in-part of application No. 11/539,475, filed on Oct. 6, 2006.
Application No. 11/539,475 is a continuation-in-part of application No. PCT/US06/13660, filed on Apr. 11, 2006.
Application No. PCT/US06/13660 is a continuation-in-part of application No. PCT/US06/13502, filed on Apr. 11, 2006.
(60) Provisional application No. 60/670,573, filed on Apr. 11, 2005.
(60) Provisional application No. 60/670,541, filed on Apr. 11, 2005.

(51) Int. Cl. A61K 31/765; A61P 19/06

(52) U.S. Cl. 424/78.17

(57) ABSTRACT

Disclosed is a method for lowering elevated uric acid levels in a patient having a plasma level of uric acid (pUAc) of more than about 6 mg/dL, the method consisting of administering to said patient an intravenous injections every 2 to 4 weeks of about 8 mg PEG-uricase.

### CROSS REFERENCE TO RELATED APPLICATIONS

This application is a continuation-in-part of U.S. Ser. No. 11/539,475, filed Oct. 6, 2006, which is a continuation-in-part of PCT/US2006/013660 and PCT/US2006/013502, both of which were filed Apr. 11, 2006, and which claim priority and benefit of U.S. Provisional Application Ser. Nos. 60/670,573, filed Apr. 11, 2005 and 60/670,541, filed Apr. 11, 2005 respectively. The disclosure of PCT/US2006/013660 and PCT/US2006/013502 as well as U.S. Provisional Application Ser. Nos. 60/670,573 and 60/670,541 are hereby incorporated by reference herein in their entireties.

### FIELD OF THE INVENTION

The invention relates to methods for lowering elevated uric acid levels using intravenous injections of PEG-Uricase.

### BACKGROUND OF THE INVENTION

Throughout this application, various publications are referenced within the text. The disclosures of these publications in their entireties are hereby incorporated by reference into this application in order to more fully describe the state of the art as known to those skilled therein as of the date of the invention described and claimed herein.

When less uric acid is excreted than is produced, plasma urate concentration (pUAc) rises and may exceed the limit of solubility (˜7 mg/dL or 0.42 mM), causing the deposition of monosodium urate (MSU) in tissues. In susceptible individuals intra-articular MSU crystals trigger inflammatory attacks of gout (Becker M A: Hyperuricemia and gout. In: The Metabolic and Molecular Bases of Inherited Disease. Edited by Scriver C R, Beaudet A L, Sly W S, Valle D, 8th edn. New York: McGraw-Hill; 2001: 2513-2535; Terkeltaub R A: Clinical practice. Gout. N Engl J Med 2003, 349(17):1647-1655; and Wortmann R L et al.: Gout and Hyperuricemia. In: Kelley's Textbook of Rheumatology. Edited by Ruddy S, Harris E D, Jr., Sledge C B, 6th edn. St. Louis: W.B. Saunders; 2001: 1339-1371).

Blocking urate production by inhibiting xanthine oxidase, or promoting renal urate excretion, can prevent further MSU crystal accumulation in tissues if plasma urate concentration is maintained below 6 mg/dL (Li-Yu J et al. J Rheumatol 2001, 28(3):577-580; Perez-Ruiz F, et al. Arthritis Rheum 2002, 47(4):356-360; and Shoji A, et al. Arthritis Rheum 2004, 51(3):321-325). If hyperuricemia is poorly controlled, gout may become chronic, leading to arthropathy, nephropathy, and various complications of tophi. Conventional therapy may be less effective at this stage since expanded tissue stores may only slowly be depleted by blocking new urate production, particularly if urate excretion is impaired by renal insufficiency, or by concomitant therapy with diuretics or cyclosporine.

Most mammals can convert uric acid to the more soluble compound allantoin. This metabolic route of elimination is inoperative in humans owing to mutation of the urate oxidase (uricase) gene during evolution. Parenteral uricase derived from Aspergillus flavus (Rasburicase, Sanofi Synthelabo) is effective in preventing acute uric acid nephropathy in patients with malignancies (Coiffier B, et al. J Clin Oncol 2003, 21(23):4402-4406; and Goldman S C, et al. Blood 2001, 97(10):2998-3003). This and other uricase preparations have been used with apparent benefit to treat small numbers of patients with refractory gout (Kissel P, et al. Nature 1968, 217:72-74; London M, et al. Science 1957, 125:937-938; Montagnac R, et al. Nephrologie 1990, 11(4):259; Moolenburgh J D, et al. Clin Rheumatol 2005:1-4; Mourad G, et al. Presse Med 1984, 13(42):2585; and Richette P, et al. Nature Clinical Practice Rheumatology 2006, 2(6):338-342). However, no clinical trials for this indication have been reported, and a relatively short circulating life and potential immunogenicity have limited their wider application for treating gout.

Attaching the inert polymer polyethylene glycol (PEG) to proteins can extend their circulating life and diminish immune recognition (Abuchowski A, et al. J Biol Chem 1977, 252(11):3582-3586; Harris J M, et al. Nat Rev Drug Discov 2003, 2(3):214-221; Veronese F M, et al. Adv Drug Deliv Rev 2002, 54(4):453-456. The development of a PEGylated recombinant mammalian uricase for treating gout is being pursued (Kelly S J, et al. J Am Soc Nephrol 2001, 12:1001-1009). In an initial Phase I clinical trial, 13 subjects with severe gout and mean pUAc >11 mg/dL received single subcutaneous (SC) injections of 4 to 24 mg of PEG-uricase (Ganson N J, et al. Arthritis Res Ther 2005, 8(1):R12). Within 7 days, pUAc fell by a mean of ˜8 mg/dL, and normalized in 11 subjects. At doses of 8-24 mg, mean pUAc remained <6 mg/dL at 21 days post-injection. Although very effective, SC-injected PEG-uricase caused transient local pain and was slowly absorbed. It was also rapidly cleared in 5 subjects who developed antibodies that, unexpectedly, reacted with PEG rather than with the uricase protein. Three of the latter subjects had allergic reactions that began at the injection site at 8-9 days post-injection (Ganson N J, et al. Arthritis Res Ther 2005, 8(1):R12).

### SUMMARY OF THE INVENTION

The present invention provides a method for lowering elevated uric acid levels in a patient having a plasma level of uric acid (pUAc) of more than about 6 mg/dL, said method comprising administering to said patient an intravenous injections every 2 to 4 weeks of about 8 mg PEG-uricase.

Other objects and advantages of the present invention will become apparent from the following description and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C show pharmacokinetics and pharmacodynamics of intravenous PEG-uricase. FIG. 1A shows the plasma uricase activity (pUox, circles) and plasma uric acid concentration (pUAc, triangles) following a single IV infusion of 8 mg of PEG-uricase. Values plotted are the means ±sd for 4 subjects. FIG. 1B shows the relationship of PEG-uricase dosage to the maximum level of plasma uricase activity (pUox, Cmax, circles) and minimum plasma uric acid concentration (pUAc, Cmin, triangles) achieved after single IV infusions. Values plotted are the means ±sd for 4 subjects. FIG. 1C shows the relationship between PEG-uricase dosage and Area Under Concentration curve (AUC) for plasma uricase activity (pUox, circles) and plasma uric acid concentration (pUAc, triangles) after single intravenous (IV) infusions (solid lines), or single subcutaneous (SC) injections (longer dashed lines). The values for SC administration are calculated from data obtained in a previous study (Ganson N J, et al. Arthritis Res Ther 2005, 8(1):R12). For each parameter, AUC was calculated from data obtained for 21 d post infusion or injection. AUC units for pUox =mU/mL·hr; AUC units for pUAc =mg/dL·hr. Values plotted are the means for 4 subjects treated at each dose level. The horizontal fine dashed line indicates the theoretical AUC value that would be obtained if pUAc was constant at 6 mg/dL for 21 d.

FIG. 2 shows the relationship between plasma uric acid concentration (mg/dL, left axis, open circles), and the ratio of uric acid:creatinine in urine (right axis, solid circles, dashed line). Upper panels show data for individual study subjects who received IV infusions of 4 mg PEG-uricase; lower panels show data for subjects who received 8 mg infusions.

FIG. 3 shows ELISAs for IgG antibodies to PEG-uricase (upper panel) and to 10 K mPEG (lower panel) in 24 Phase I study subjects. Plasma samples tested were obtained prior to (day 0), and on days 14, 21, and 35 following a single intravenous infusion of the indicated dose of PEG-uricase. Horizontal lines above the X-axis represents the mean +3 sd values obtained in each ELISA for a panel of normal individuals, which serve as the cutoff for scoring samples from study subjects as positive or negative in the ELISA. The ‘Key’ legend indicate whether a subject has “sero-converted” from a negative to a positive ELISA response after infusion of PEG-uricase (black numbers, negative; bold red numbers, positive). In 2 cases where the day 0 sample exceeded the cutoff value (italicized orange numbers), the result was considered indeterminate.

FIG. 4A-4D show the Structural PK-PD Models Tested. (4A) Model 1—Direct inhibitory Emax model (4B) Model 2—Direct inhibitory sigmoidal Emax model (4C) Model 3—Indirect inhibitory Emax model (4D) Model 4—Indirect inhibitory sigmoidal Emax model. C, PEG-uricase concentration; CL, systemic clearance; Emax, maximum inhibitory capacity; γ, Hill factor; IC₅₀, concentration of PEG-uricase where 50% of maximal inhibitory effect is attained; Kin, rate of formation of uric acid; Kout, rate of elimination of uric acid; Vc, central volume of distribution.

FIG. 5 shows the Final PK-PD Model, where C=PEG-uricase concentration, Vc=Central volume of distribution for PEG-uricase, CL=Systemic clearance of PEG-uricase, Emax=Maximum inhibitory capacity, and IC₅₀=Concentration of PEG-uricase where 50% of maximal inhibitory effect is attained.

FIG. 6A-6J show the relationships between Covariates and PK Parameters. (6A) Vc vs. weight; (6B) CL vs. weight; (6C) Vc vs. Ideal Body Weight; (6D) CL vs. Ideal Body Weight; (6E) Vc vs. age; (6F) CL vs. age; (6G) Vc vs. Gender; (6H) CL vs. Gender; (6I) Vc vs. Race; and (6J) CL vs. Race.

FIGS. 7A-7O show the relationships between Covariates and PD Parameters. (7A) Emax vs. weight; (7B) IC₅₀ vs. weight; (7C) Baseline uric acid vs. weight; (7D) Emax vs. ideal body weight; (7E) IC₅₀ vs. ideal body weight; (7F) Baseline uric acid vs. ideal body weight; (7G) Emax vs.age; (7H) IC₅₀ vs. age; (7I) Baseline uric acid vs. age; (7J) Emax vs. gender (K) IC₅₀ vs. gender; (7L) Baseline uric acid vs. gender; (7M) Emax vs race; (7N) IC₅₀ vs. race; and (7O) Baseline uric acid vs. race.

FIG. 8 shows an example of Individual Fitted Pharmacokinetic-Pharmacodynamic Profile.

FIGS. 9A-9D show the results of Quality of Fit. (9A) Predicted vs. observed PEG-uricase concentrations; (9B) Weighted residuals vs time for PEG-uricase; (9C) Predicted vs. observed uric acid concentrations; and (9D) Weighted residuals vs time for uric acid.

FIG. 10A-10D show the Monte Carlo Simulations. (10A) Predicted concentrations following administration of 8 mg PEG-uricase every 2 weeks for 18 months; (10B) Predicted concentrations following administration of 8 mg PEG-uricase every 2 weeks for 6 months then every 4 weeks for 12 months; (10C) Predicted concentrations following administration of 8 mg PEG-uricase every 4 weeks for 18 months; and (10D) Predicted concentrations following administration of 8 mg PEG-uricase every 4 weeks for 6 months then every 2 weeks for 12 months.

### DETAILED DESCRIPTION OF THE INVENTION

It has been surprisingly discovered that IV PEG-uricase is superior to subcutaneous (SC) enzyme injections in achieving a more rapid, significant, and prolonged lowering of plasma urate concentration (pUAc), and it greatly reduces urinary uric acid excretion. Furthermore, while single infusions of PEG-uricase induced anti-PEG antibodies in some subjects, no allergic reactions were encountered.

The dosage of IV injections of PEG-Uricase is from about 0.5 to about 12 mg, preferably from about 4 to about 12 mg. PEG-Uricase may also be administered by IV injection in dosages between 4 and 8 mg, or between 8 and 12 mg.

The uricase used in PEG-Uricase may comprise a mammalian uricase amino acid sequence truncated at the amino terminus or the carboxy terminus or both the amino and carboxy termini by about 1-13 amino acids and may further comprise an amino acid substitution at about position 46. The truncated uricase may further comprise an amino terminal amino acid, wherein the amino terminal amino acid is alanine, glycine, proline, serine, or threonine as described in co-pending PCT/US2006/013660 and U.S. provisional application Ser. No. 60/670,573, which are hereby incorporated herein by reference in their entireties.

### Phase I Study

A Phase I study of single IV infusions of PEG-uricase in 24 hyperuricemic subjects with severe gout was completed as indicated in the Examples below. A Phase I trial of subcutaneously injected enzyme in 13 such patients was previously carried out (Ganson N J, et al. Arthritis Res Ther 2005, 8(1):R12). Both trials examined PEG-uricase at doses of 4, 8 and 12 mg in groups of 4 subjects, and monitored study parameters for 21 days after dosing (lower doses of 0.5, 1 and 2 mg were also tested in the present trial). The results of these two trials have established suitable conditions of dose and the frequency and route of administration for use in later stages in the clinical evaluation of PEG-uricase.

PEG-uricase is intended for patients with poorly controlled hyperuricemia who have failed other forms of therapy. Subjects in the present (and previous) trial had recent or continuous clinical manifestations of gout; 75% had tophi. In none had a therapeutic serum urate concentration (<6.0 mg/dL) been adequately maintained with available urate lowering medications (whether this was due to noncompliance, lack of efficacy at prescribed dosages, or to drug intolerance was not considered in selecting subjects for this Phase I trial). The mean pUAc prior to infusion of PEG-uricase was 10.9±0.5 mg/dL. In most subjects uricase activity was detectable in plasma for the full 21-day post-infusion period of observation.

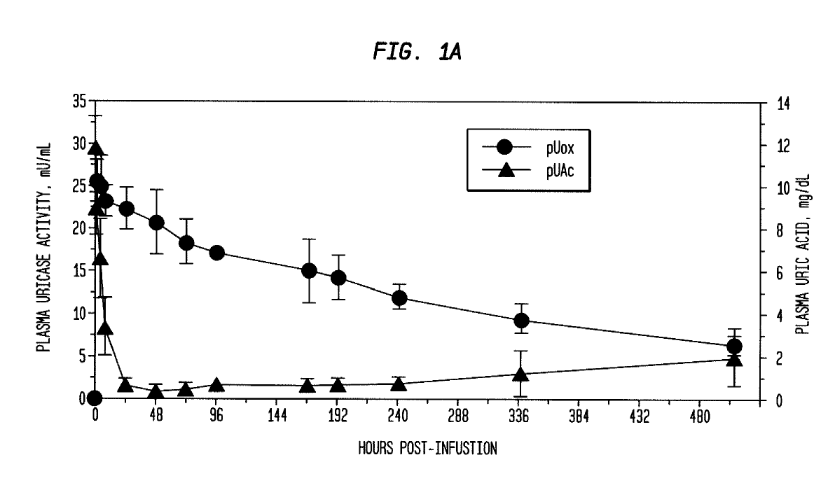

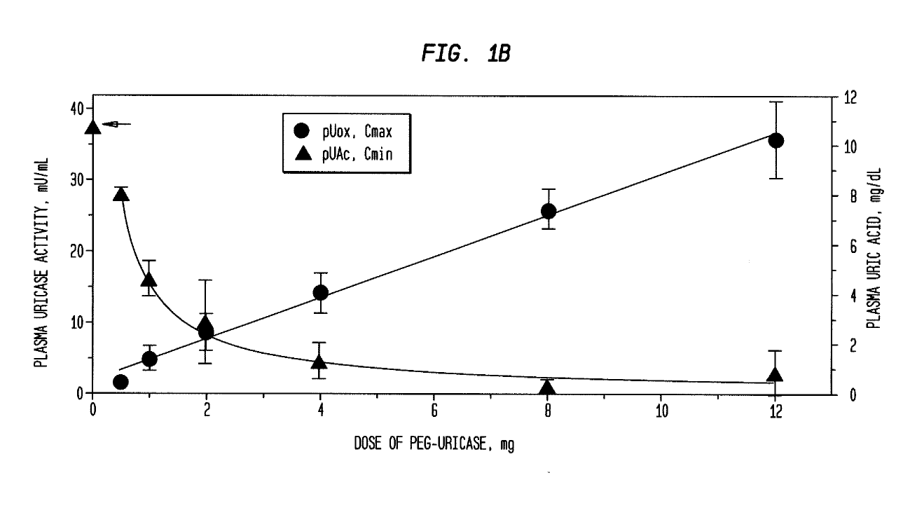

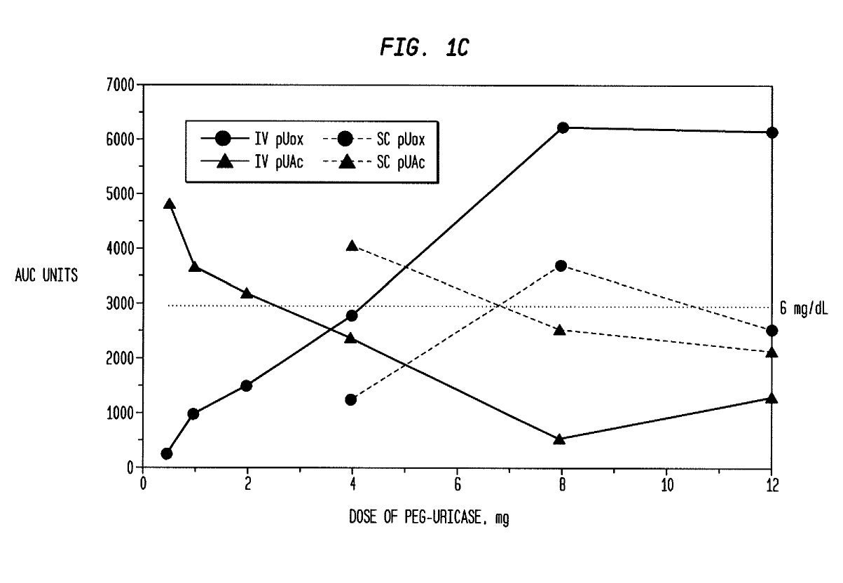

The pharmacodynamic results of most interest were obtained with doses of 4, 8, and 12 mg, which were most effective in lowering urate levels in plasma and urine. The onset of action was rapid: mean pUAc fell to <2 mg/dL within 24 h post-infusion, and the maximum decline in pUAc from baseline averaged 10.3 mg/dL at 24-72 h. Of importance, the AUC for the entire 21-d period post-infusion was equivalent to maintaining a constant pUAc of 4.7, 1.2, and 2.7 mg/dL at PEG-uricase doses of 4, 8, and 12 mg, respectively. This prolonged lowering of pUAc to below the therapeutic target of 6 mg/dL is more impressive in view of the level of disease and inadequate response of these subjects to conventional therapy. Comparison of AUC data for both pUox and pUAc at doses of 4-12 mg obtained in the present and previous trial (FIG. 1C) clearly indicates the superior bioavailability and efficacy of IV versus SC PEG-uricase.

Single infusions of PEG-uricase in the 4 to 12 mg range also markedly lowered the UAc:Cr ratio in urine in parallel with the effect on pUAc. Measuring UAc:Cr in spot urine samples might provide an alternative way to monitor the response to PEG-uricase therapy, with the advantage of not requiring measures to inhibit PEG-uricase, as is necessary to accurately assess the effect on serum/plasma urate concentration. The ability to greatly reduce or eliminate uric acid excretion could be of particular benefit in patients with uric acid nephrolithiasis, which may complicate chronic gout.

The results with single infusions of PEG-uricase indicated that doses of about 4 to 12 mg at intervals of 2 to 4 weeks would maintain a therapeutic response.

Infused PEG-uricase was generally well tolerated, and all adverse events were classified as mild or moderate; their frequency was unrelated to dose. The only adverse events considered possibly related to PEG-uricase were gout flares and arthralgia in 14 subjects. The present study population reported frequent gout flares prior to the trial, and reducing serum urate concentration with other drugs has also been associated with an increased incidence of gout flares (Becker M A, et al. N Engl J Med 2005, 353(23):2450-2461; Emmerson B T, N Engl J Med 1996, 334:445-451; and Yamanaka H, et al. Adv Exp Med Biol 1998, 431:13-18.

IgG antibodies to PEG-uricase, in most cases of the IgG2 subclass and specific for PEG, developed in 9 out of 24 Phase I subjects. There was a trend toward more rapid terminal clearance of PEG-uricase in the antibody-positive subjects. However, in contrast to the cutaneous reactions in 3 of 5 antibody-positive subjects treated with PEG-uricase (Ganson N J, et al. Arthritis Res Ther 2005, 8(1):R12.), no allergic reactions occurred in the present trial. Also, whereas several subjects developed local pain and swelling within a few hours of receiving SC injections of PEG-uricase, there were no infusion reactions in the present study.

Hydrogen peroxide (H₂O₂), a byproduct of urate oxidation, was postulated to cause inflammation at the SC injection site, which, along with the slow absorption of SC PEG-uricase, may have contributed to antibody development and late allergic reactions (Ganson N J, et al. Arthritis Res Ther 2005, 8(1):R12). In considering the absence of infusion and delayed allergic reactions in the present study, it is significant that infused PEG-uricase is largely confined to the intravascular space where very high levels of catalase in red cells can efficiently eliminate H₂O₂ produced in plasma. Also, since H₂O₂ is only generated by PEG-uricase as urate is oxidized, the rate of intravascular H₂O₂ production would decrease markedly within 24 h of the infusion of PEG-uricase if pUAc is maintained close to or below 1 mg/dL.

The results herein indicate that IV doses of about 0.5 to 12 mg of PEG-uricase administered every 2-4 weeks will maintain plasma urate well below 6 mg/dL, and will be effective in rapidly reducing tophus size. PEG-uricase may also be used in patients with chronic gout and hyperuricemia that is poorly controlled with existing therapies.

### Phase II Study

A Phase II study was conducted as described hereinbelow in details in the Examples to examine a continuum of PEG-uricase doses that was expected to maintain uric acid levels below 6 mg/dL, at 2 dosing intervals (that is, 4 mg every 2 weeks, 8 mg every 2 weeks, 8 mg every 4 weeks and 12 mg every 4 weeks) in order to determine the optimal dose and dosing frequency to control uric acid levels in the population of patients with treatment-failure gout. Intravenous administration was selected in order to minimize immune response. This Phase II study was designed to evaluate the efficacy, pharmacokinetic profile and safety of PEG-uricase administered repeatedly by intravenous infusion.

A compartmental model was developed to simultaneously describe serum concentrations of PEG-uricase and plasma concentrations of uric acid. A one-compartment model with linear elimination best described the PK of PEG-uricase. The PK-PD model included an inhibitory Emax effect (as a function of PEG-uricase concentration) resulting in a decrease in uric acid levels with increasing PEG-uricase concentrations.

Weight was the only significant covariate for the PK parameters CL and Vc. There were no covariates that influenced PD parameters in a significant manner.

According to this model, PEG-uricase was generally able to suppress uric acid concentrations up to 83%, and maximal suppression was attained at very low serum concentrations of PEG-uricase.

Based on predictive simulations performed using this model, PEG-uricase given as 2-hour IV infusions every 2 or 4 weeks at 8 mg will maintain uric acid levels well below 6 mg/dL. The long half-life of PEG-uricase supports the use of the infrequent (i.e., every 2 or 4 week) dosing regimens.

### EXAMPLE 1

### Material, Methods and Design of Phase I Clinical Study

### Investigational Drug

PEG-uricase consists of a recombinant mammalian uricase (primarily porcine, with C-terminal sequence from baboon uricase), conjugated with multiple strands of monomethoxy PEG of average molecular weight 10 kDa (10 K mPEG) per subunit of tetrameric enzyme (Kelly S J, et al. J Am Soc Nephrol 2001, 12:1001-1009; and Ganson N J, et al. Arthritis Res Ther 2005, 8(1):R12). It was manufactured by Savient Pharmaceuticals, Inc. (East Brunswick, N.J.) and supplied in vials containing 12.9 mg of PEG-uricase (233 Units, assayed as described below) in 1 mL of a phosphate buffer.

### Phase I Study Design

An open-label study was conducted in 24 adults with symptomatic gout, who were assigned sequentially to 6 cohorts of 4 subjects each, to receive single IV infusions lasting 60 minutes and containing 0.5, 1, 2, 4, 8, or 12 mg of PEG-uricase in 50 mL of saline. The protocol and consent form were approved by the Duke University Institutional Review Board. Uric acid lowering medications were withheld for 7 days prior to, and for 21 days after dosing. The primary outcomes were the pharmacokinetics and safety of PEG-uricase. Secondary outcomes were the effects of PEG-uricase on pUAc, and on the ratio of uric acid to creatinine in urine (UAc:Cr). Adverse events and changes in clinical laboratory tests were used to assess safety for 35 days after dosing. Pharmacokinetic and pharmacodynamic parameters were assessed for 21 days after dosing. The IgG antibody response to PEG-uricase was assessed prior to, and on days 7, 14, and 35 post-infusion.

### Subjects

Inclusion requirements were: age ≧18 years; symptomatic gout (tophi, chronic synovitis due to gout, or gout flare within the last 6 months); and a pUAc ≧7 mg/dL after discontinuing uric acid lowering therapy for at least 7 days. Subjects were excluded for any of the following: unstable coronary artery disease; uncontrolled hypertension; renal insufficiency requiring dialysis; baseline serum aminotransferase levels >1.5 times the upper limit of normal in the absence of known cause; organ transplantation requiring immunosuppressive therapy; requirement for corticosteroid at a dose of >10 mg of prednisone (or equivalent) within one week of dosing; continued use of uric acid lowering medications; acute gout flare at baseline; glucose-6-phosphate dehydrogenase deficiency; or previous administration of urate oxidase.

### Pharmacokinetic and Pharmacodynamic Measurements

PEG-uricase was monitored as uricase catalytic activity in plasma (pUox) as described (Ganson N J, et al. Arthritis Res Ther 2005, 8(1):R12). Results are expressed as mU per mL plasma, where 1 U=1 μmol of urate oxidized per min. Plasma urate concentration (pUAc) was measured after acidification to inactivate PEG-uricase (Ganson N J, et al. Arthritis Res Ther 2005, 8(1):R12).

### Immune Response to PEG-Uricase

ELISAs for IgG antibodies to PEG-uricase and to 10 K mPEG-glycine (10 K mPEG conjugated with glycine instead of uricase protein) were performed in the Hershfield laboratory as described (Ganson N J, et al. Arthritis Res Ther 2005, 8(1):R12]. Screening was performed on dilutions (1:20 and 1:60) of pre-dose (day 0), day 14, day 21, and day 35 plasma samples. Plasma from an antibody-positive (i.e. to both PEG-uricase and 10 K mPEG-glycine) subject identified in the previous Phase I trial of SC PEG-uricase (Ganson N J, et al. Arthritis Res Ther 2005, 8(1):R12) was used as a positive reference. A “positive” ELISA was defined as an absorbance at 405 nm (A405)>3 sd above the mean for a panel of plasma samples from naive subjects.

The IgG subclass of antibodies binding specifically to PEG-uricase immobilized on an ELISA plate was determined with mouse anti human IgG1, IgG2, IgG3, and IgG4-specific antibodies (Sigma, St. Louis, Mo.).

### Other Laboratory Studies

A routine chemistry panel, complete blood count (CBC), glucose-6-phosphate dehydrogenase, and haptoglobin were obtained at screening visit. Pregnancy was excluded by serum beta-HCG in women of child-bearing potential. C-reactive protein, erythrocyte sedimentation rate, and complement proteins C3 and C4, C1q binding assay, and CH50 were measured prior to dosing and on days 3, 10, 14 and 21 after dosing, along with a chemistry panel, CBC, and haptoglobin.

### Evaluation of Safety

Subjects were monitored for 35 d after receiving PEG-uricase. Adverse events, including gout flares, detected by study personnel, or elicited from or volunteered by study subjects were recorded. Gout flares were treated according to the judgment of the study physician.

### EXAMPLE 2

### Phase I Clinical Study Using Infusion of PEG-Uricase

A clinical study was carried out as indicated in Example 1 above. The results are indicated below.

### Subject Characteristics

The demographic and gout disease characteristics of study subjects are shown in Table 1 below. Common co-morbidities associated with gout, including obesity, hypertension, coronary artery disease, and renal stones, were distributed relatively evenly among the 6 dosing cohorts, although 3 of 4 subjects in the 4 mg cohort had type II diabetes mellitus. Mean age ranged from 41.8 y in the 2 mg, to 64.5 y in the 12 mg dose cohort. Mean body mass index ranged from 28.3 in the 2 mg, to 36.5 in the 8 mg dose cohorts.

TABLE 1
Characteristics of Phase I Trial Subjects

| | |
|---|---|
| Gender | Female 4, male 20 |
| Age (y) | 56.7 ± 12.9 (28-73) |
| Number of subjects with: | acute gout attacks, 22 (92%); chronic synovitis, 15 (62.5%); |
| | tophi, 18 (75%); nephrolithiasis, 5 (21%) |
| Body Mass Index | 32.2 ± 6.6 (23.4-49.2) |
| Serum Uric Acid* | On allopurinol (7 subjects): 6.6 ± 1.2 mg/dL (4.8-7.8) |
| | Not on allopurinol (17 subjects): 9.4 ± 0.9 mg/dL (7.2-12.3) |
| Serum Creatinine* | 1.2 ± 0.4 mg/dL (0.8-2.2) |
| Most frequent co- | hypertension, 16; diabetes, 6; osteoarthritis, 6; cardiac dysrhythmias, 6; |
| morbidities | coronary artery disease, 3. |

*Values measured prior to the drug washout period

A history of acute gout attacks was reported by 92% of subjects; flares were monoarticular in 41%, oligoarticular in 27%, and polyarticular in 32%. Chronic synovitis was present in 62.5% of subjects and 75% had tophi. At screening, the mean ±sd serum urate concentration was 9.4±0.9 mg/dL in the 17 subjects who were not receiving antihyperuricemic medication, and 6.6±1.2 mg/dL in the 7 subjects who were receiving allopurinol. After the washout period pUAc in the latter subjects rose to 8.6±0.8 mg/dL.

Pharmacokinetics and Pharmacodynamics with Single Infusions of PEG-Uricase

Prior to infusion of PEG-uricase, the mean ±sd pUAc for all 6 dose cohorts was 10.9±0.5 mg/dL (range 10.7-11.8 mg/dL); pUox was undetectable in all subjects. FIG. 1A plots these parameters following infusion of PEG-uricase for the 8 mg dose cohort, which had the highest baseline pUAc. Maximum post-infusion pUox was 26±2.8 mU/mL, and the level after 21 d was 6.5±1.1 mU/mL; the plasma half-life for pUox was 300±21 h (12.5±0.9 d). Within 24 h of dosing, mean pUAc had decreased by 11.2 mg/dL, and reached a nadir of 0.3 mg/dL at 72 h post-infusion. At 21 d, the mean pUAc was 2 mg/dL, or 9.8 mg/dL below baseline.

Maximal pUox (Cmax) increased linearly with dose of PEG-uricase (FIG. 1B). The nadir value for pUAc (Cmin) declined steeply at doses of 0.5-2 mg and was <1.5 mg/dL at doses of 4-12 mg, with an average reduction of 10.3 mg/dL (range 9.5-11.5 mg/dL) below baseline. Cmin for pUAc occurred at 48-72 h post-infusion for the 1-8 mg dose cohorts, and at 24 h in 3 of 4 subjects in the 12 mg cohort. Mean half-life for pUox for the 6 dose cohorts was 220±77 h (9.2±3.2 d), with a range from 163-332 h (6.4-13.8 d). The volume of distribution for PEG-uricase ranged from about 5,000 to 10,000 mL.

Area-under-concentration curve (AUC) parameters for pUox and pUAc were inversely related to one another, and were each proportional to the dose of PEG-uricase between 0.5 and 8 mg (FIG. 1C). Doses of 4, 8, and 12 mg resulted in AUC values for pUAc equivalent to maintaining constant pUAc levels of 4.7, 1.2, and 2.7 mg/dL, respectively, for 21 d post-infusion. The superior bioavailability and efficacy of IV compared with SC administration is evident.

### Effect of PEG-Uricase on Urinary Excretion of Uric Acid

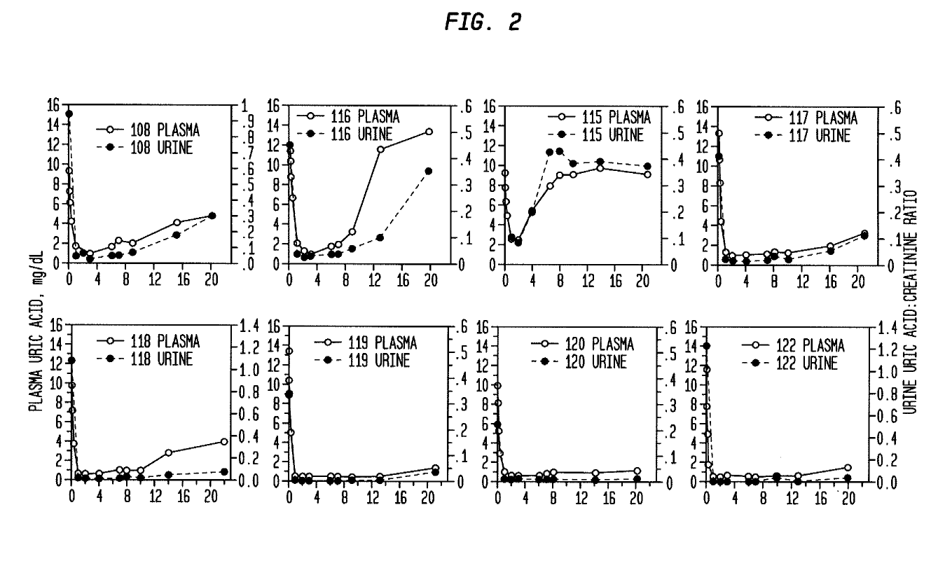

Infusion of PEG-uricase markedly reduced renal uric acid excretion, as indicated by a decline in UAc:Cr in spot urine samples. This is illustrated for the 4 mg and 8 mg dose cohorts in FIG. 2, which also shows coordinate effects on pUAc and urinary UAc:Cr.

### Immune Response to IV PEG-Uricase

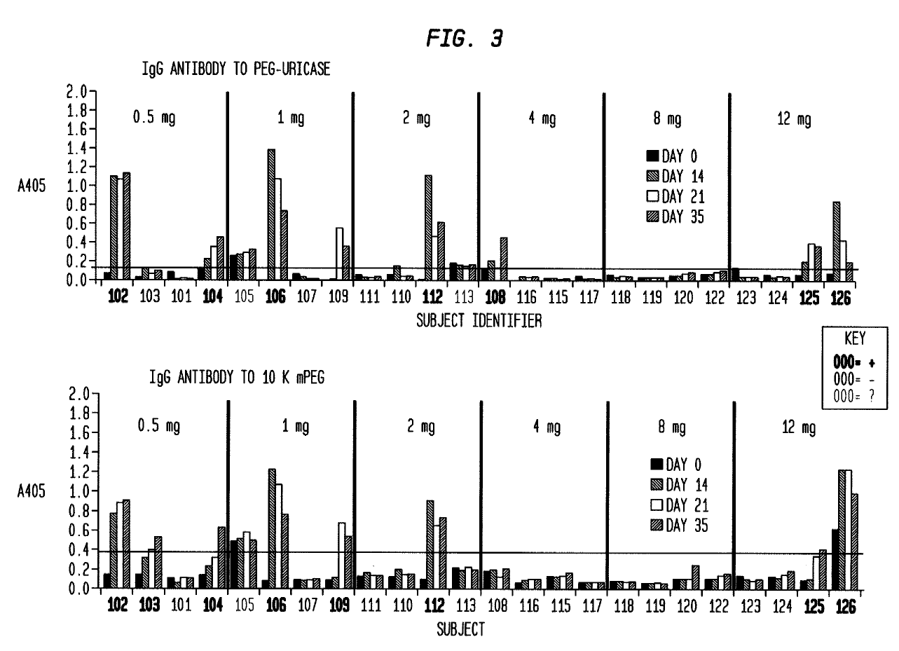

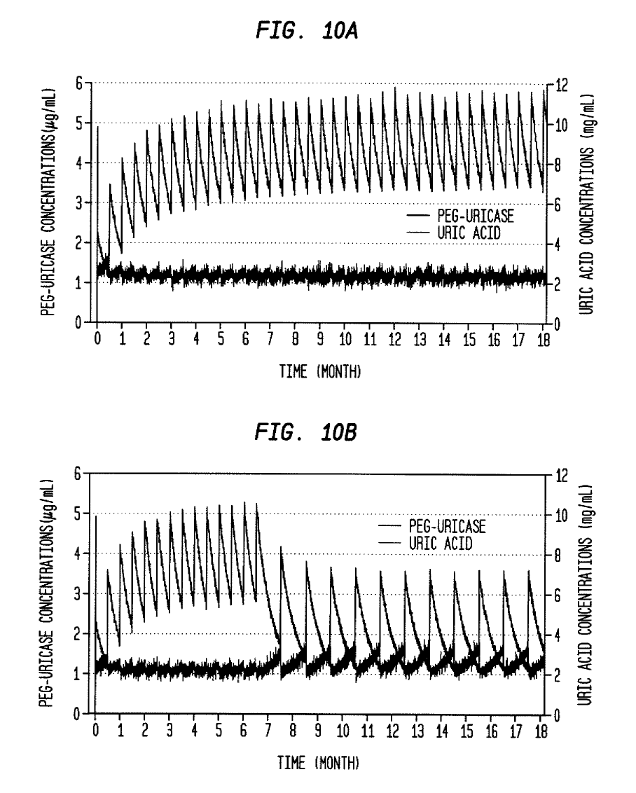

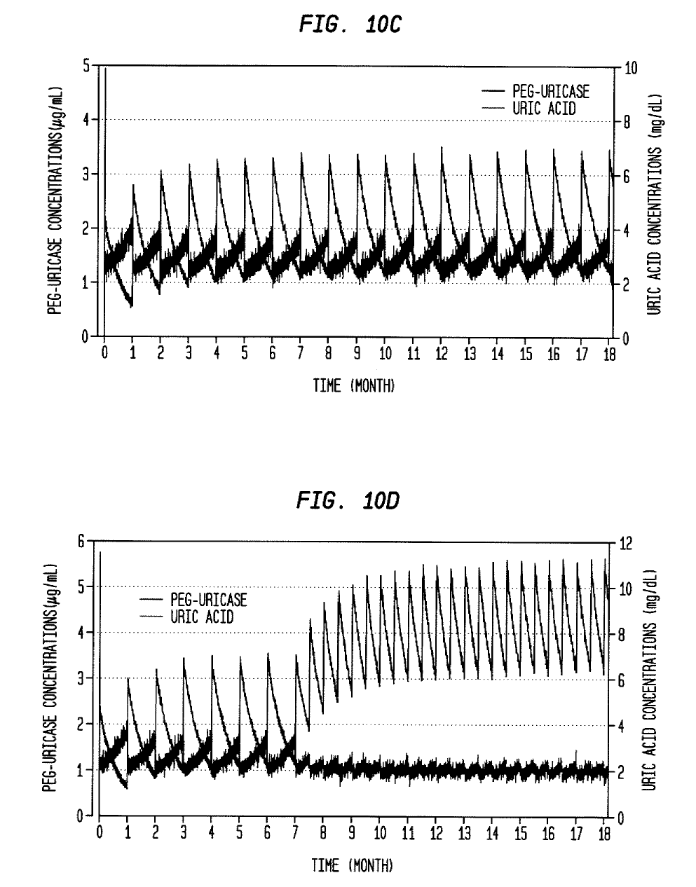

Prior to treatment, 22 of the 24 Phase I subjects had negative ELISAs for IgG antibodies to both PEG-uricase (FIG. 3A) and 10 K mPEG (FIG. 3B). Both ELISAs remained negative in 13 of these subjects when tested again at d 14, 21, and 35 post-infusion. The other 9 subjects developed positive ELISAs, in 7 cases for both antigens, and in 2 cases for one or the other antigen. Studies not presented showed that antibodies that reacted with PEG-uricase and 10 K mPEG were of the IgG2 subclass, or in some cases both IgG2 and IgG3.

Antibody testing was inconclusive in 2 Phase I subjects who had positive ELISAs at baseline, which did not change significantly after infusion of PEG-uricase. Plasma from subject 113 reacted with PEG-uricase, but not with 10 K mPEG, whereas plasma from subject 105 was positive with both antigens. Of interest, in this latter subject pUox was only detected transiently during the first 24 h after infusion, compared with detectable pUox levels for 7 to 21 d in the 3 other subjects in the 1 mg dose cohort (data not shown).

In a previous trial, IgG antibody to PEG-uricase appeared at 7 days after SC administration, about when pUox was maximal owing to slow absorption from the injection site (Ganson N J, et al. Arthritis Res Ther 2005, 8(1):R12). In antibody-positive subjects pUox declined rapidly from this peak to undetectable levels by day 10-14 post infusion. In the present study, a trend towards more rapid terminal clearance of infused PEG-uricase was indicated by the finding that pUox could last be detected at 11.0±6.0 d (range 4-21 d) in ELISA-positive, vs. 16.1±5.9 d (range 4-22 d) in ELISA-negative subjects (p=0.06 in a 2-tailed T test).

Evidence of antibody-mediated clearance was also obtained when antibody-positive subject 109 in the 1 mg dose cohort was given a second infusion of 8 mg of PEG-uricase about a year after his exposure in the Phase 1 trial. The ELISA to PEG-uricase had become negative prior to the second infusion, but became strongly positive again by 7 d after dosing. For 48 h after infusion, pUox levels were consistent with this 8 mg dose of PEG-uricase, causing pUAc to decline by 10 mg/dL. However, on d 7 post-infusion, pUox was undetectable (whereas it was measurable for 9 d after the first 1 mg dose), and pUAc had returned to the pre-infusion level. No allergic phenomena were associated with this anamnestic antibody response.

### Safety and Tolerability

All 24 Phase I subjects completed the study. There were no serious adverse events or clinically important changes in laboratory results. Twenty-two Phase I subjects experienced 66 adverse events, all of mild to moderate severity (Table 2). Twenty-one of these were considered possibly related to study treatment; all but one were gout flares, the other being arthralgia. The risk of an adverse event was similar in each dosing cohort. None of the subjects experienced infusion reactions.

TABLE 2
Adverse Events

| | |
|---|---|
| Total subjects | 24 |
| Number of Subjects with Events | 22 (92%) |
| Gout | 14 (58.3%) |
| Blood pressure increased | 2 (8.3%) |
| Arthralgia | 4 (16.7%) |
| Back pain | 2 (8.3%) |
| Diarrhoea nos | 2 (8.3%) |
| Dyspepsia | 2 (8.3%) |
| Dizziness | 3 (12.5%) |
| Upper respiratory tract infection nos | 2 (8.3%) |
| Insomnia | 2 (8.3%) |
| Total subjects | 24 |
| Other (occurring in one subject): | 10 |
| hyperglycemia, hypokalemia, anemia, | |
| headache, pruritus, sweating, aesthenia, | |
| peripheral edema, fever, herpes simplex, | |
| hypotension | |

The most common adverse event was acute gout flare (20 flares in 14 study subjects). The number of subjects experiencing a gout flare was 0 in the 0.5 mg cohort; 2 in 1 mg and 12 mg cohorts; 3 in the 2 mg and 4 mg cohorts; and 4 in the 8 mg cohort. The mean time to onset of an initial gout flare was 13.6 d (range 2-32 d). No relationship was observed between PEG-uricase dose and the time to an initial gout flare. Non-steroidal anti-inflammatory drugs, colchicine, or oral corticosteroids were used as prophylaxis or treatment for gout flares in 23/24 subjects.

### EXAMPLE 3

### Phase II Subjects Materials and Methods

### Subjects, Study Design and Treatments

This was a randomized, open-label, multi-center, parallel study of multiple intravenous doses of PEG-uricase, administered via infusion, in 41 patients with symptomatic gout. Outpatients of either gender were included in the study if they were at least 18 years of age, diagnosed with symptomatic gout refractory to conventional therapy or unable to tolerate conventional therapy, hyperuricemic (screening serum uric acid >8 mg/dL) and willing and able to give informed consent and adhere to visit/protocol schedules. Women of childbearing potential must have had a negative serum pregnancy test and were required to use an approved birth control method during their participation in the protocol.

Patients were excluded from the study if there was unstable coronary artery disease or uncontrolled hypertension, history of end stage renal disease requiring dialysis, history of liver disease (defined by baseline serum transaminase elevation >3× the upper limit of normal in the absence of any other known cause), immunosuppressive therapy required by organ transplantation, concurrent use of prednisone at a dose >10 mg qd (or equivalent) at or within one week before dosing, concurrent use of uric acid-lowering agents, prior treatment with PEG-uricase or other recombinant uricase, an acute gout flare within one week prior to first treatment with PEG-uricase (exclusive of chronic synovitis/arthritis) requiring use of medication which violates the protocol, glucose-6-phosphate dehydrogenase deficiency, a history of anaphylactic reaction to a recombinant protein or porcine derivatives, lactation, administration of an investigational drug within 4 weeks prior to study drug administration or plans to take an investigational agent during the study, known allergy to urate oxidase or PEGylated products or any other medical or psychological condition which, in the opinion of the investigator, might create undue risk to the patient or interfere with the patient's ability to comply with the protocol requirements.

TABLE 3
summarizes patient demographics

| | Patients | | |
|---|---|---|---|
| | Demographics | Mean (CV %) | Median (Range) |
| ALL | Age (years) | 58.1 (23.7%) | 61.0 (22-83) |
| (N = 41, 35 Men | Body Weight (kg) | 97.5 (28.3%) | 91.2 (47.2-167) |
| and 6 Women) | Height (cm) | 176 (6.42%) | 178 (155-196) |

The clinical design of the study consisted of patients assigned to one of 4 dosing groups of intravenous PEG-uricase: 4 mg every 2 weeks, 8 mg every 2 weeks, 8 mg every 4 weeks or 12 mg every 4 weeks. Each patient received 3 or 6 intravenous infusions of PEG-uricase infused over 30 to 60 minutes. Treatment duration was approximately 12 weeks.

PEG-uricase pharmacokinetics was determined through analysis of serum samples drawn at protocol specified time-points. PEG-uricase pharmacodynamics was determined by measuring uric acid levels in plasma during each treatment cycle. Table 4 outlines the blood sampling schedule.

TABLE 4
Blood Sampling Schedule

| Regimen | | | | | |
|---|---|---|---|---|---|
| | Dose | Cycle | | | |
| Frequency | (mg) | 1 | 2 | 3* | 6 |
| Every 2 | 4 | Pre-dose | Pre-dose | Pre-dose | Pre-dose |
| weeks | and 8 | Post-dose: 1.5, 4, | Post-dose: | | Post-dose: 1.5, 4, |
| | | 6, 48, 96, 192 h | 192 h | | 6, 48, 96, 192, |
| | | | | | 360, 528, 696, |
| | | | | | 1344 h |
| Every 4 | 8 | Pre-dose | Pre-dose | Pre-dose | N/A |
| weeks | and 12 | Post-dose: 1.5, 4, | Post-dose: 192, | Post-dose: 1.5, 4, | |
| | | 6, 48, 96, 192, | 360, 528 h | 6, 48, 96, 192, | |
| | | 360, 528 h | | 360, 528, 696, | |
| | | | | 1344 h | |

*Includes Cycles 4 and 5 for treatments given every 2 weeks.

In addition, the urine uric acid:creatinine ratio were determined at various times following PEG-uricase dosing. Safety and tolerability were evaluated throughout the study, including clinical laboratory evaluations and the occurrence of clinical adverse events. Immune response to PEG-uricase was assessed by measuring antibody levels to PEG-uricase. The previous uric acid-lowering treatment could have been reinstituted 56 days after the last dose administration. Including screening and washout activities, a patient's participation was expected to be up to 18 weeks.

### Analytical Assays

Serum PEG-uricase was measured as PEG-uricase activity. This assay comprised an enzymatic/fluorescent reaction, with an analytical range of 0.6 to 10 μg/mL. Plasma uric acid was measured using fluorescent assay, with an analytical range of 5 to 100 μg/mL. Antibody levels were also analyzed using an ELISA assay.

### Population Pharmacokinetic-Pharmacodynamic Analysis

Actual dosing and sampling times were used for the compartmental modeling of serum PEG-uricase and plasma uric acid. Only patients with detectable concentrations of active PEG-uricase and uric acid following drug administrations were included in the analysis. Samples that were hemolyzed or that did not meet acceptance criteria were not included in the PK analysis. Serum concentrations of PEG-uricase that were below the limit of quantitation were set to missing for the population PK modeling. Plasma concentrations of uric acid that were below the limit of quantitation were set to 0.25 mg/dL (half of the lower limit of quantitation) for the population PD modeling.

Multiple compartmental models were first constructed for serum PEG-uricase concentrations using ADAPT-II® (D'Argenio D Z, Schumitzky A. ADAPT-II Users Manual. Biomedical Simulations Resource, University of Southern California, Los Angeles, 1997.) The model discrimination process was performed by comparing the AIC (Akaike Criterion) values and by looking at pertinent graphical representations of goodness of fit (e.g. fitted and observed concentrations versus time, weighted residuals versus observed values). All relevant pharmacokinetic parameters were calculated and reported.

Following the determination of the final structural model in ADAPT-II® , population PK-PD analysis was performed with IT2S® using the relevant pharmacokinetic parameters obtained in ADAPT-II® as prior estimates (Forrest A, Hawtoff J, Egorin M J: Evaluation of a New Program for Population PK/PD Analysis Applied to Simulated Phase I Data. Clinical Pharmacology and Therapeutics 49(2):153, 1991). The impact of covariates could be evaluated by coding the variable into the equations defining the compartmental model or by assessing them graphically in a post-hoc analysis.

Covariates investigated for inclusion in the model included: age, gender, race, body weight, ideal body weight and antibody levels.

Once the structural PK model for serum PEG-uricase was determined, various PK-PD models were evaluated using ADAPT-II®. Based on the criteria described above, the model that best fit the uric acid data was selected to conduct population PK-PD analyses in IT2S®.

All serum PEG-uricase concentrations and plasma uric acid concentrations were fitted using a weighting procedure of Wⱼ=1/σⱼ² where the variance σⱼ² was calculated for each observation using the equation Sⱼ²=(a+b×yⱼ)² where a and b are the intercept and slope of each variance model. The slope is the residual variability proportional to each concentration, and the intercept is the additional component of the residual variability. These residual variability parameter estimates were updated iteratively during the population PK analysis until stable values were found.

Overall, a total of 498 serum concentrations of PEG-uricase and 769 plasma concentrations of uric acid were fitted simultaneously from a total of 40 patients. Only patients with detectable concentrations of serum PEG-uricase and plasma uric acid were included in the population PK-PD analysis. Patient No. 9004 did not have available PEG-uricase concentrations therefore this patient was excluded from the PK/PD analysis.

### Monte Carlo Simulations

Once the final PK-PD model including covariates was selected, Monte Carlo simulations were performed in order to predict serum PEG-uricase and plasma uric acid concentrations following the administration of various dosing regimens. Several dosing strategies were evaluated in order to determine the dosage required to maintain uric acid levels below 6 mg/dL.

Monte Carlo simulations were performed using a population of 160 patients receiving 4 different regimens which were the proposed Phase 3 dose regimens. The infusion duration of 2 hours was selected in order to further minimize potential immune responses. The first regimen consisted of a 2-hour IV infusion of PEG-uricase (8 mg) administered every 2 weeks for 18 months. The second regimen consisted of an 8 mg 2-hour IV infusion of PEG-uricase administered every 2 weeks for 6 months followed by administration every 4 weeks for another 12 months. The third regimen consisted of an 8 mg 2-hour IV infusion of PEG-uricase administered every 4 weeks for a total of 18 months. Finally, the last regimen comprised an 8 mg 2-hour IV infusion of PEG-uricase administered every 4 weeks for 6 months, followed by administration every 2 weeks for 12 months.

### EXAMPLE 4

### Phase II Clinical Study Using Infusion of PEG-Uricase

A clinical study was carried out as indicated in Example 2 above. The results are indicated below.

### Model Discrimination

One- and two-compartment models with linear elimination processes were evaluated to describe the serum pharmacokinetics of PEG-uricase. In addition, non-linear elimination processes were also tested (Michaelis-Menten elimination).

The structural model that best described the pharmacokinetics of PEG-uricase was a one-compartment model with linear elimination. An inter-occasion variability (IOV) factor was added to the clearance to try to improve the quality of fit. The IOV term allowed the systemic clearance (CL) of PEG-uricase on the last cycle (i.e., a second “occasion”) to be slightly different than that observed on the first cycle within patients. However, the IOV term did not improve the quality of fit in a statistically significant manner and therefore the one-compartment model was retained as the best model.

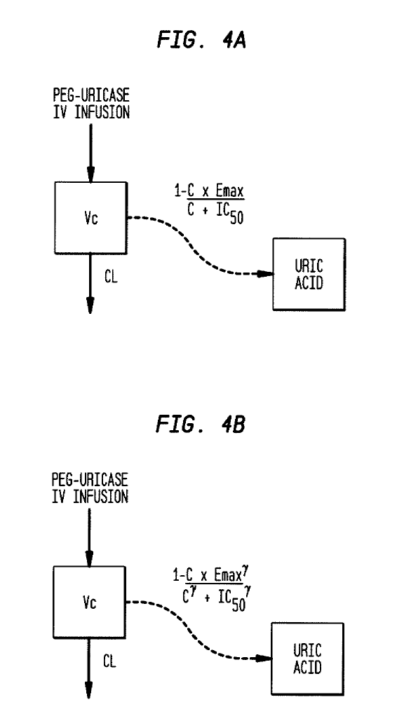

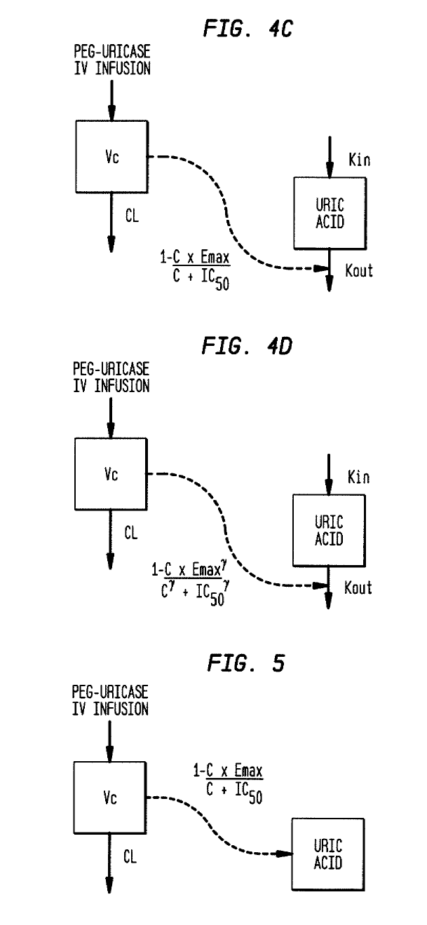

Using the pre-determined one-compartment PK model, various PD models were tested using ADAPT-II®. In each of the models tested, the PD of the biomarker (plasma uric acid) was correlated to the PK of PEG-uricase. Selection of the best PK-PD model was based on minimization of the AIC as well as visual inspection of the fit. FIGS. 4A-4D depict some of the models that were evaluated.

In both the direct and indirect models tested, the sigmoidal Emax model did not provide a statistically significant improvement in comparison with the Emax model. Similarly, the indirect models did not provide a superior quality of fit in comparison with the direct models. Therefore, the model that best described the PK-PD relationship between serum PEG-uricase and plasma uric acid was Model 1. In this model, uric acid levels decreased with increasing PEG-uricase concentrations according to the following equation:

$$UA ( t ) = {UA}_{baseline} \times [ 1 - \frac{E max \times C}{{IC}_{50} + C} ]$$

where UA(t)=observed uric acid level at any given time; UA_{baseline}=baseline uric acid level before PEG-uricase administration; C=PEG-uricase concentration; Emax=maximal inhibitory capacity of PEG-uricase; and IC₅₀=concentration of PEG-uricase where 50% of maximal inhibitory effect is attained. The final PK-PD model is depicted in FIG. 5.

### Covariate Testing

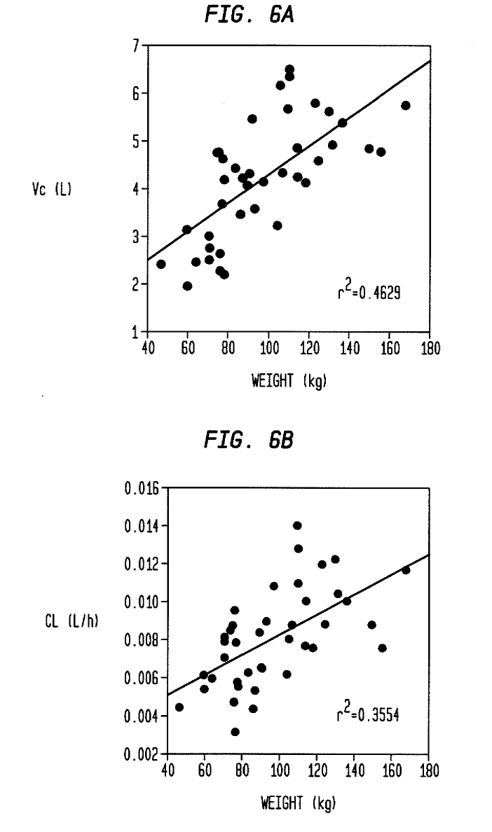

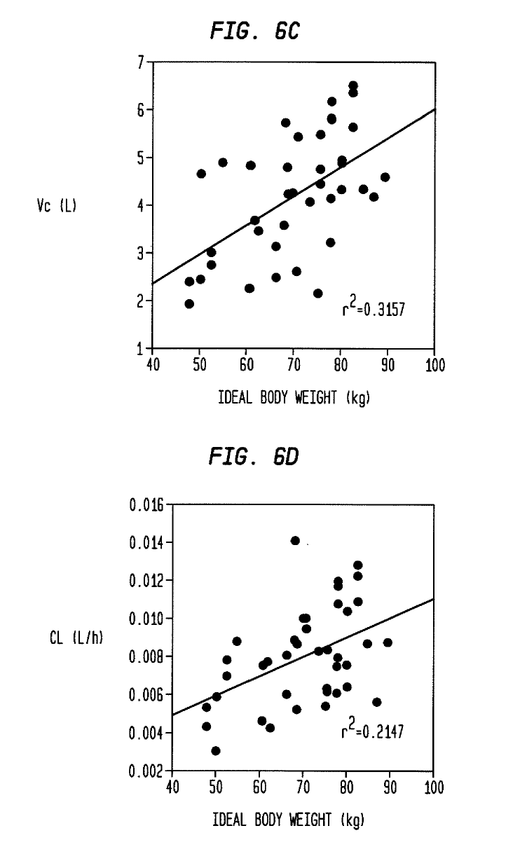

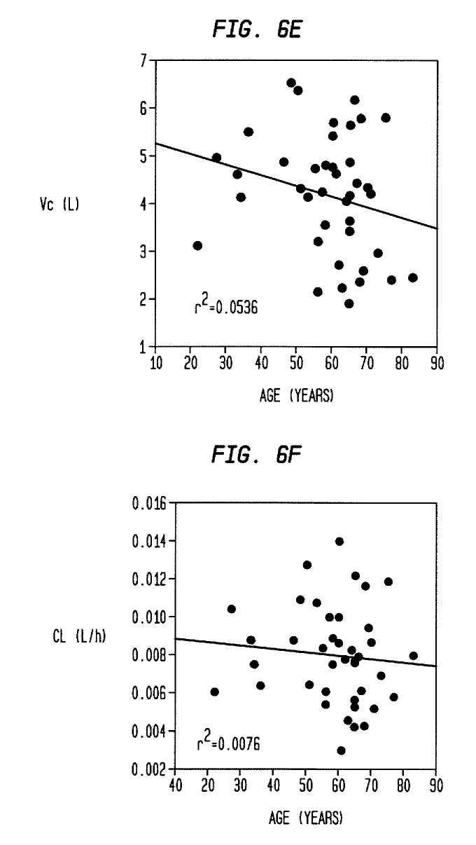

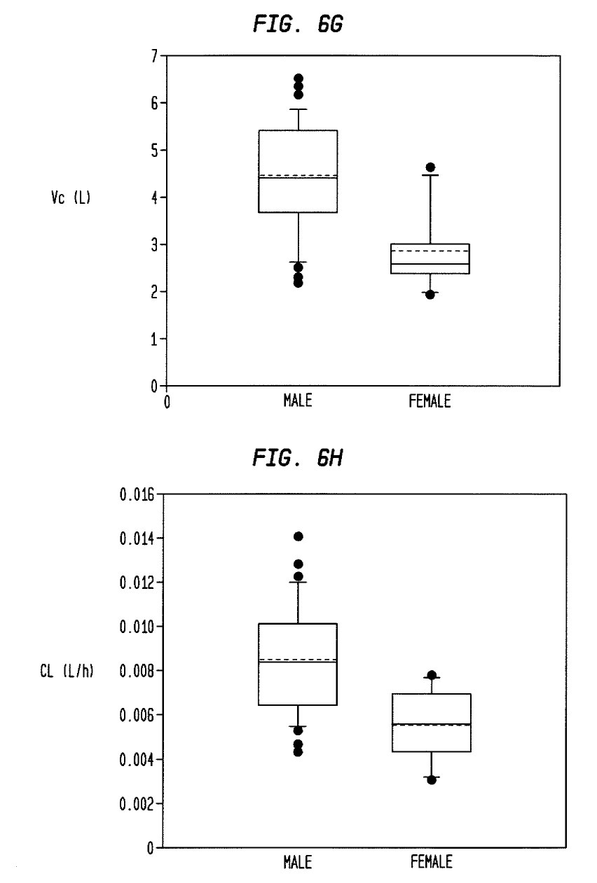

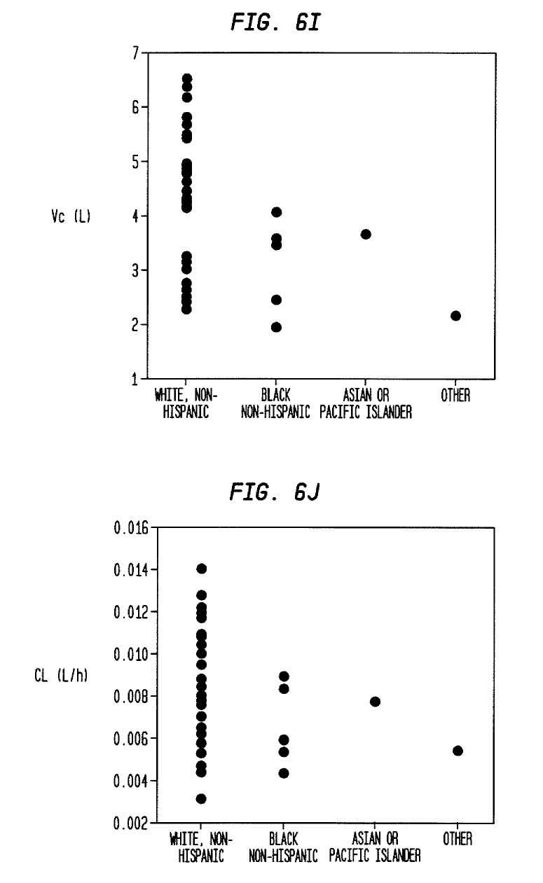

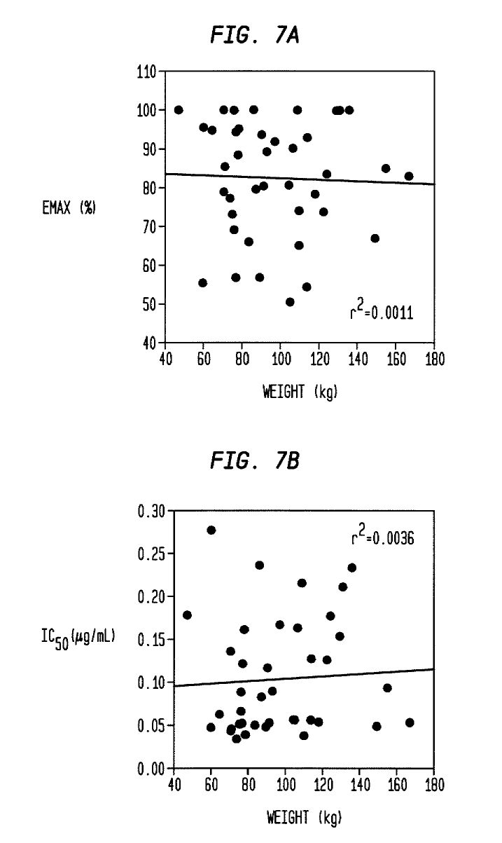

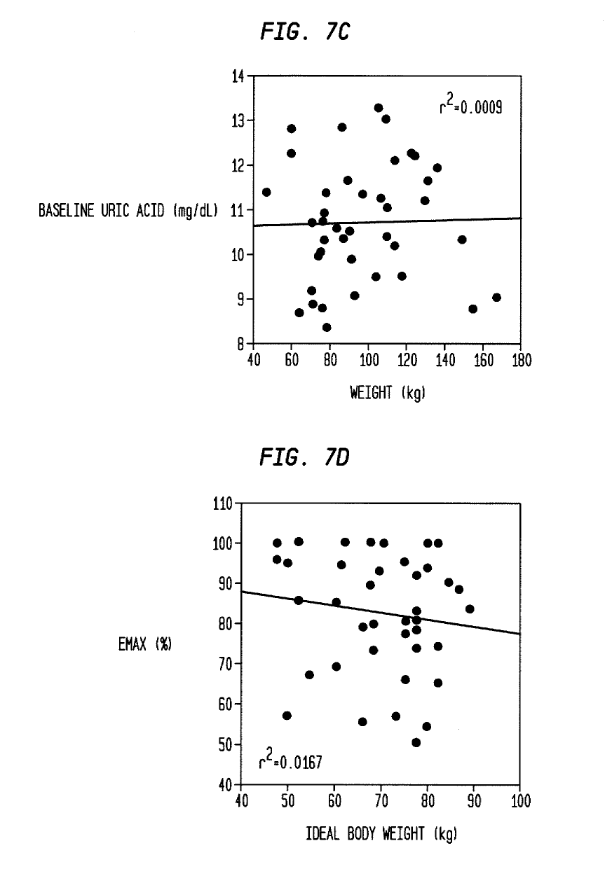

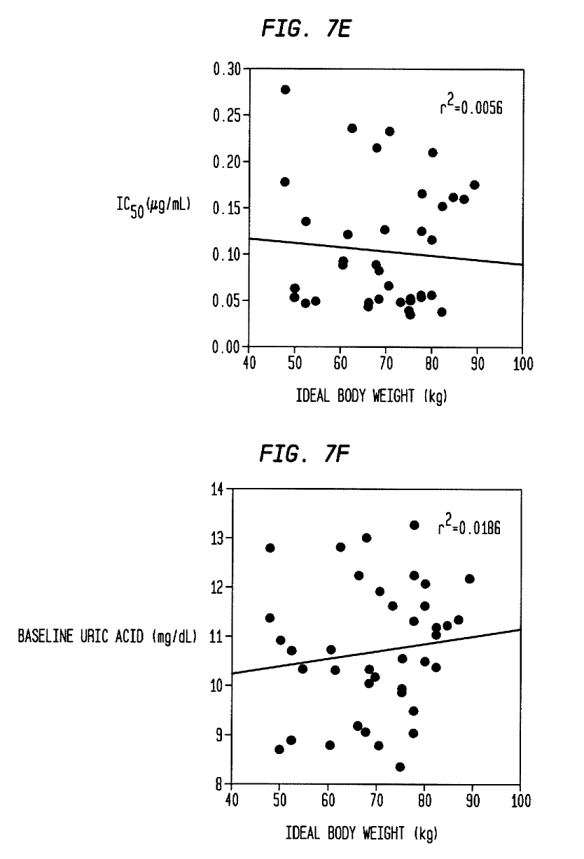

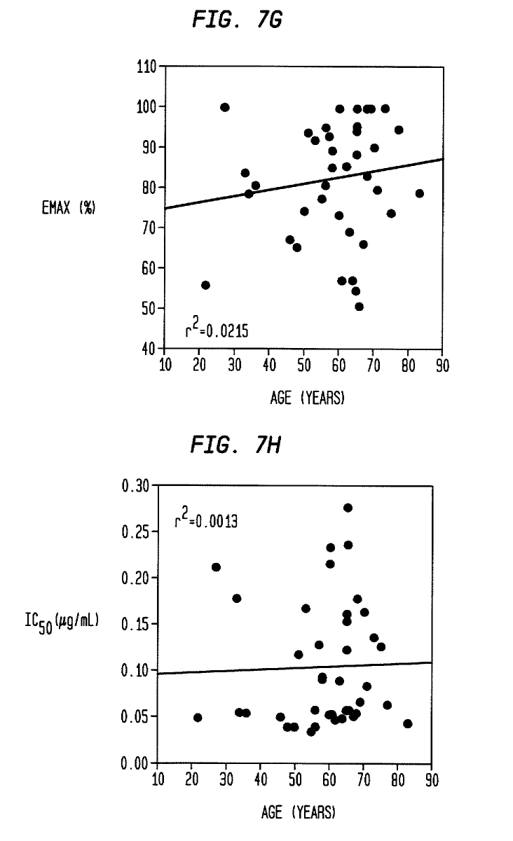

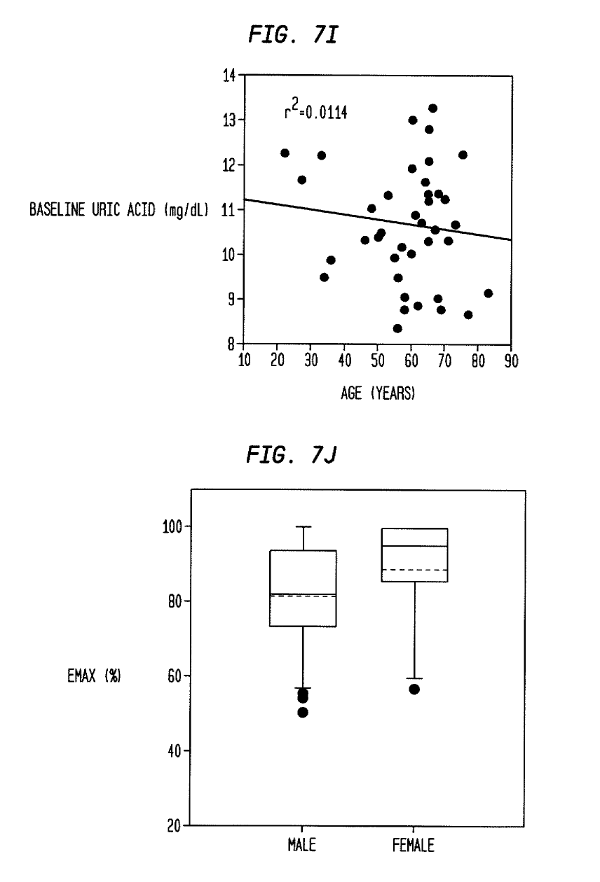

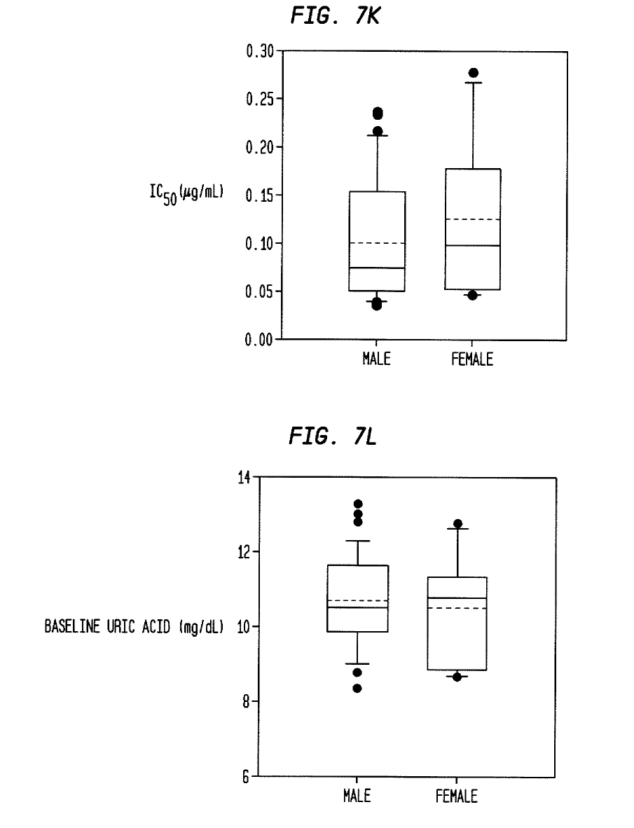

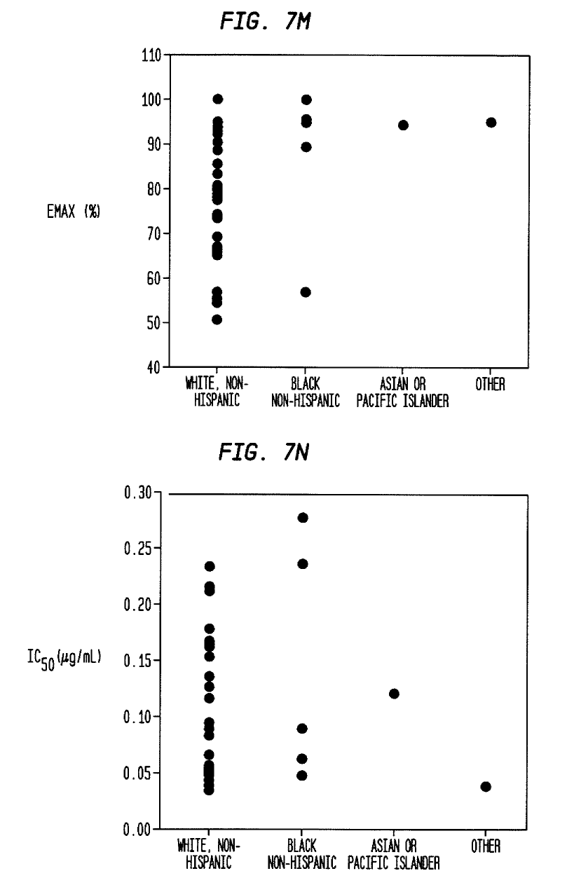

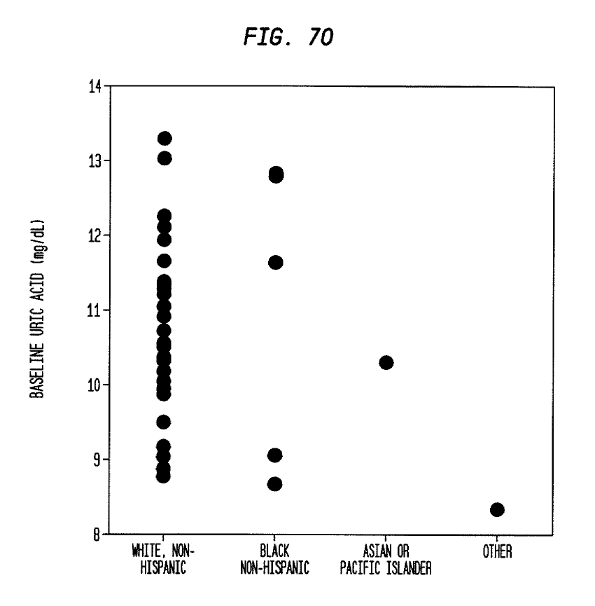

Relationships between various covariates and PK and PD parameters are depicted in FIGS. 6A-6J and 7A-7L. The relationships between the PK parameters and body weight were similar to those observed between the PK parameters and ideal body weight. However, based on correlation coefficients (r² values), the relationship between body weight and the PK parameters CL and Vc was more important, therefore the dose was adjusted for body weight in the PK model. Weight did not influence any of the PD parameters in a significant manner. Because height was already taken into account in the calculation of ideal body weight, it was not tested as a separate covariate.

Neither age nor gender was a significant covariate for PK or PD parameters. Most patients were white, non-hispanic and therefore it was difficult to assess the effect of race on the PK and PD parameters of PEG-uricase. In addition, the “Asian or pacific islander” and “other” categories were only represented by one patient each. However, results suggest that there were no significant trends related to race.

Antibody levels were determined for each of the patients at various timepoints following dosing. Because each patient's antibody levels changed as a function of time, it was necessary to take this into account when developing the PK and PD model and assessing the impact of this covariate on the PK and PD parameters of PEG-uricase.

In order to assess the potential influence of fluctuating antibody levels on the PK and PD parameters of PEG-uricase, two different approaches were used. The first method included antibody level in the model as a covariate that changed with time. For timepoints where no antibody level was available, it was assumed the antibody level was the same as the previously detected level. In other words, the “last observation carried forward” (LOCF) technique was applied to this method (Hu C, Sale M E. A joint model for nonlinear longitudinal data with informative dropout. J Pharmacokinet Pharmacodyn 2003; 30 (1): 83-103.). The second approach consisted of modeling the antibody levels in ADAPT-II® and including the fitted antibody levels as a covariate in the PK-PD model.

Various models using both approaches were then tested to determine whether or not the inclusion of antibody levels improved the quality of fit. Although antibody levels were well fitted by an indirect model (with a rate of formation, Kin, and a rate of elimination, Kout) that was a function of PEG-uricase concentrations, none of the models tested improved the quality of fit of the PK parameters of PEG-uricase. Therefore, antibody level was not retained in the final model as a covariate.

### Quality of Fit and PK Parameter Estimates

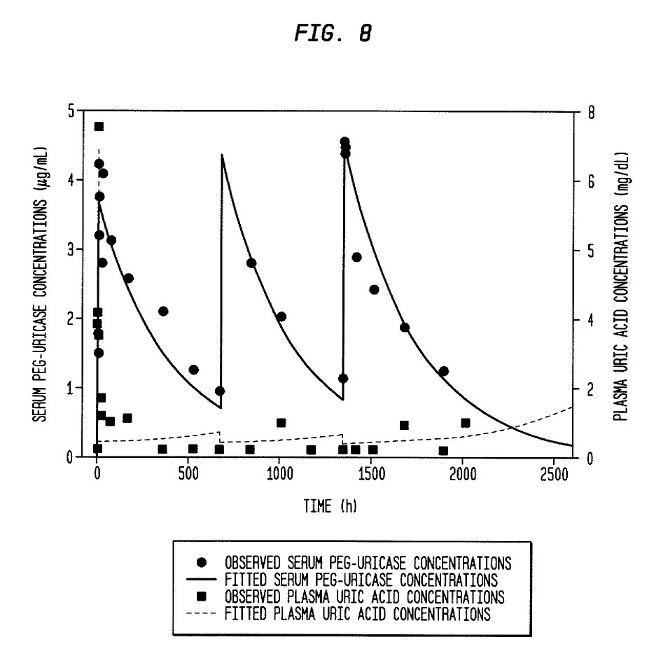

FIG. 8 illustrates an example of a fitted pharmacokinetic-pharmacodynamic profile.

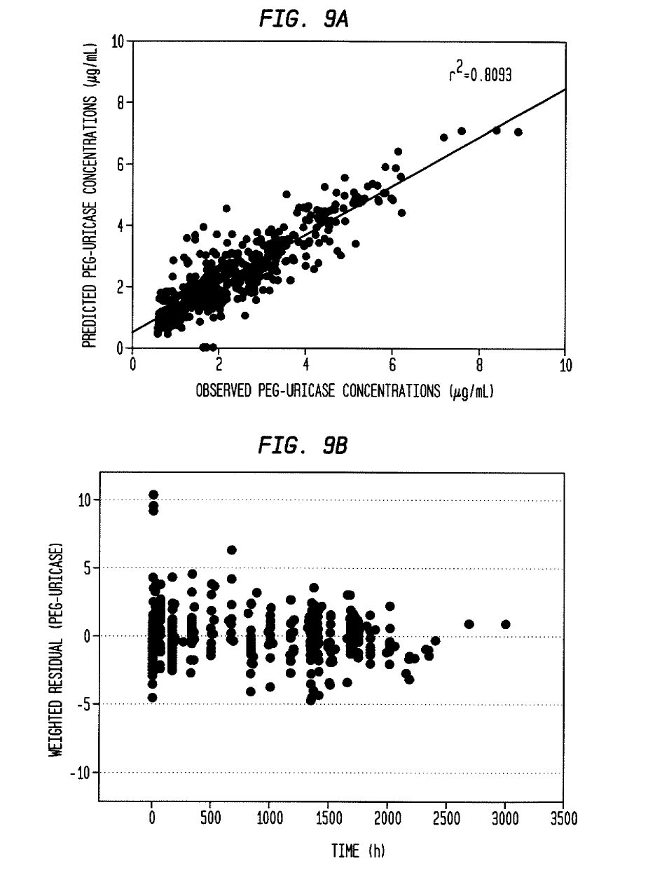

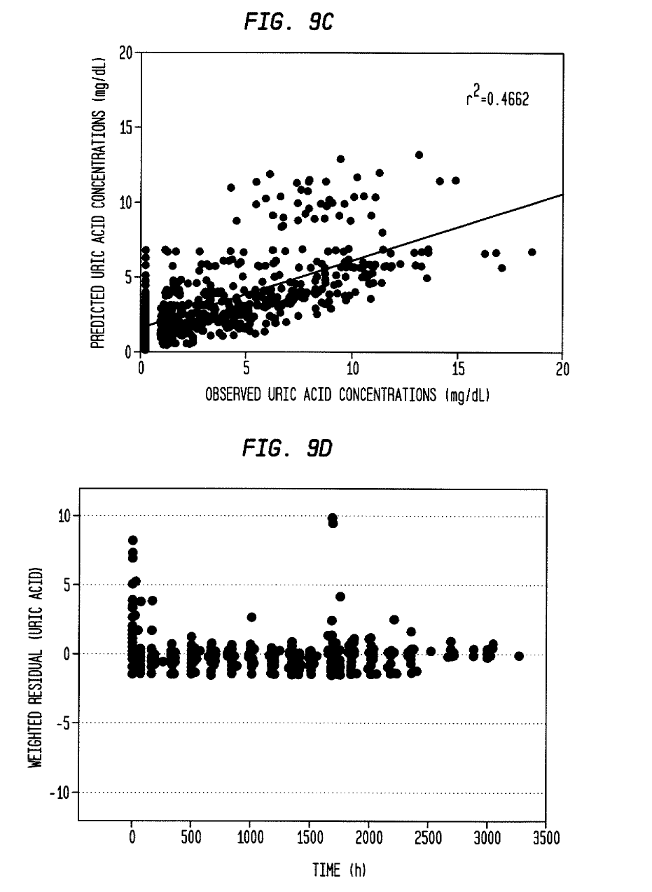

Results for each quality of fit are depicted in FIGS. 9A-9D. Overall, individual plasma concentrations of PEG-uricase and uric acid were adequately fitted using this model, with r² values of 0.8093 and 0.4662, respectively. Weighted residuals for PEG-uricase and uric acid did not show any bias over time.

Mean values for the pharmacokinetic and pharmacodynamic parameters of PEG-uricase along with inter-individual variability (CV %), median and range are presented in Table 5.

TABLE 5
Post-hoc Population PK-PD Parameters of PEG-
uricase and Uric Acid (n = 40)

| Parameters | Mean (CV %) | | Median (Range) | |
|---|---|---|---|---|
| CL (L/h/kg) | 0.0000854 | (28.2%) | 0.0000839 | (0.0000392-0.000136) |
| CL (L/month/kg) | 0.0615 | (28.2%) | 0.0604 | (0.0282-0.0976) |
| Vc (L/kg) | 0.0449 | (21.2%) | 0.0468 | (0.0278-0.0639) |
| Half-life (h) | 404 | (44.2%) | 349 | (170-1049) |
| Baseline uric acid (mg/dL) | 10.7 | (12.5%) | 10.6 | (8.34-13.3) |
| Emax (%) | 82.5 | (18.1%) | 84.3 | (50.4-100) |
| IC₅₀ (μg/mL) | 0.104 | (65.1%) | 0.0740 | (0.0338-0.278) |
| IC₉₀ (μg/mL)* | 0.932 | (65.1%) | 0.666 | (0.304-2.50) |

| | |
|---|---|
| Residual Variability PEG-uricase (%) | 25.8% |
| Residual Variability Uric Acid (%) | 83.7% |

*IC₉₀: Concentration of PEG-uricase where 90% of maximal inhibitory effect on uric acid levels is attained.

### Monte Carlo Simulations

Results of the Monte Carlo Simulations are presented in FIGS. 10A-10D. Overall, the predicted uric acid levels remained well below 6 mg/dL for all treatment regimens. This suggests that a regimen of 8 mg given every 2 weeks or every 4 weeks would be effective in maintaining uric acid concentrations at target levels.

Therefore, The simplest model that best described the serum concentrations of PEG-uricase was a one-compartment model with linear elimination. The effect of serum PEG-uricase on the plasma concentrations of uric acid was best described by a direct inhibitory Emax model. Body weight was the only covariate influencing the PK parameters CL and Vc.

The residual variabilities reported for PK and PD parameters represent the variability that is not explained by the model, including interindividual variability, the experimental “noise” of the analytical method and errors arising from the pharmacokinetic modeling itself. Significant variability in the PEG-uricase and uric acid concentrations was observed, which could explain the residual variability values obtained above, as well as the elevated inter-individual variability associated with certain PK and PD parameters.

Of all the covariates investigated for inclusion in the PK/PD model, weight was the only significant covariate that influenced the PK parameters CL and Vc. The absence of any significant covariates on the PD parameters suggests that the effect of PEG-uricase on the plasma uric acid levels is not influenced by factors such as gender, age or race. Similarly, body weight was not found to affect the PD parameters directly, although weight influenced PK parameters.

The mean value for the central volume of distribution of PEG-uricase was 0.0449 L/kg. This value corresponds to the approximate plasma volume of a human subject weighing 70 kg (3000 mL/70 kg =0.0429 L/kg) (Davies B, Morris T. Physiological Parameters in Laboratory Animals and Humans. Pharmaceutical Research 1993; 10 (7): 1093-95).

The mean Emax value for PEG-uricase was 82.5% with low inter-subject variability, indicating that most patients' uric acid concentrations were decreased by approximately 83%. This was consistent with the marked suppression of uric acid observed in the population, since many of the uric acid values fell below the limit of quantitation after single and repeated intravenous infusions of PEG-uricase.

The mean IC₅₀ value of 0.104 μg/mL indicates that very low levels of PEG-uricase are able to provide 50% of the maximal suppression of uric acid levels. This value is all the more remarkable since it is below the lower limit of quantitation (LLOQ) of serum PEG-uricase (0.6 μg/mL). Similarly, the mean IC₉₀ value of 0.932 μg/mL demonstrates that levels of PEG-uricase slightly above the LLOQ are sufficient to attain most of the maximal inhibitory effect (90% of the effect) of PEG-uricase.

The half-life of PEG-uricase in serum was approximately 2 weeks long, ranging from 170 to 1049 hours. This characteristic of the compound supports the use of a less frequent dosing regimen.

Accordingly, a compartmental model was developed to simultaneously describe serum concentrations of PEG-uricase and plasma concentrations of uric acid. A one-compartment model with linear elimination best described the PK of PEG-uricase. The PK-PD model included an inhibitory Emax effect (as a function of PEG-uricase concentration) resulting in a decrease in uric acid levels with increasing PEG-uricase concentrations.

Weight was the only significant covariate for the PK parameters CL and Vc. There were no covariates that influenced PD parameters in a significant manner.

According to this model, PEG-uricase was generally able to suppress uric acid concentrations up to 83%, and maximal suppression was attained at very low serum concentrations of PEG-uricase.

Based on predictive simulations performed using this model, PEG-uricase given as 2-hour IV infusions every 2 or 4 weeks at 8 mg will maintain uric acid levels well below 6 mg/dL. The long half-life of PEG-uricase supports the use of the infrequent (i.e., every 2 or 4 week) dosing regimens.

The invention has been described in terms of preferred embodiments thereof, but is more broadly applicable as will be understood by those skilled in the art. The scope of the invention is only limited by the following claims.

What is claimed is:

1. A method for lowering elevated uric acid levels in a patient having a plasma level of uric acid (pUAc) of more than about 6 mg/dL, said method comprising administering to said patient an intravenous injection every 2 to 4 weeks of about 8 mg PEG-uricase.

2. The method of claim 1, wherein said administration causes uric acid levels in said patient to drop below 6 mg/dL.

3. The method of claim 1, wherein said intravenous injections occur every 2 weeks.

4. The method of claim 1, wherein said intravenous injections occur every 4 weeks.

5. The method of claim 1, wherein said patient is suffering from gout.

6. The method of claim 5, wherein said gout is refractory.

7. The method of claim 1, wherein said gout is chronic or tophaceous.

8. The method of claim 1, wherein said PEG-uricase comprises a mammalian uricase amino acid sequence truncated at the amino terminus or the carboxy terminus or both the amino and carboxy termini by about 1-13 amino acids.

9. The method of claim 8, wherein said truncated amino acid sequence further comprises an amino terminal amino acid, wherein the amino terminal amino acid is selected from the group consisting of alanine, glycine, proline, serine and threonine.
